SEQUENCE LISTING (43 sequences)

SEQ ID NO: 1 - protein, 37 aa, synthetic construct
  REGION 1..37: note = CGRP-alpha
ACDTATCVTHRLAGLLSRSGGVVKNNFVPTNVGSKAF

SEQ ID NO: 2 - protein, 37 aa, synthetic construct
  REGION 1..37: note = CGRP-beta
ACNTATCVTHRLAGLLSRSGGMVKSNFVPTNVGSKAF

SEQ ID NO: 3 - protein, 5 aa, synthetic construct
  REGION 1..5: note = CDR-H1 Vyepti
GYYMN

SEQ ID NO: 4 - protein, 16 aa, synthetic construct
  REGION 1..16: note = CDR-H2 Vyepti
VIGINGATYYASWAKG

SEQ ID NO: 5 - skipped

SEQ ID NO: 6 - protein, 111 aa, synthetic construct
  REGION 1..111: note = VH Vyepti
EVQLVESGGGLVQPGGSLRLSCAVSGIDLSGYYMNWVRQAPGKGLEWVGVIGINGATYYASWAKGRFTISRDNSKTTVYLQMNSLRAEDTAVYFCARGDIWGQGTLVTVSS

SEQ ID NO: 7 - protein, 440 aa, synthetic construct
  REGION 1..440: note = HC Vyepti
EVQLVESGGGLVQPGGSLRLSCAVSGIDLSGYYMNWVRQAPGKGLEWVGVIGINGATYYASWAKGRFTISRDNSKTTVYLQMNSLRAEDTAVYFCARGDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDARVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - protein, 441 aa, synthetic construct
  REGION 1..441: note = HC-2 Vyepti
EVQLVESGGGLVQPGGSLRLSCAVSGIDLSGYYMNWVRQAPGKGLEWVGVIGINGATYYASWAKGRFTISRDNSKTTVYLQMNSLRAEDTAVYFCARGDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDARVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 13 aa, synthetic construct
  REGION 1..13: note = CDR-L1 Vyepti
QASQSVYHNTYLA

SEQ ID NO: 10 - protein, 7 aa, synthetic construct
  REGION 1..7: note = CDR-L2 Vyepti
DASTLAS

SEQ ID NO: 11 - protein, 13 aa, synthetic construct
  REGION 1..13: note = CDR-L3 Vyepti
LGSYDCTNGDCFV

SEQ ID NO: 12 - protein, 113 aa, synthetic construct
  REGION 1..113: note = VL Vyepti
QVLTQSPSSLSASVGDRVTINCQASQSVYHNTYLAWYQQKPGKVPKQLIYDASTLASGVPSRFSGSGSGTDFTLTISSLQPEDVATYYCLGSYDCTNGDCFVFGGGTKVEIKR

SEQ ID NO: 13 - protein, 219 aa, synthetic construct
  REGION 1..219: note = LC Vyepti
QVLTQSPSSLSASVGDRVTINCQASQSVYHNTYLAWYQQKPGKVPKQLIYDASTLASGVPSRFSGSGSGTDFTLTISSLQPEDVATYYCLGSYDCTNGDCFVFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 5 aa, synthetic construct
  REGION 1..5: note = CDR-H1(Fremanezumab)
NYWIS

SEQ ID NO: 15 - protein, 19 aa, synthetic construct
  REGION 1..19: note = CDR-H2 (Fremanezumab)
EIRSESDASATHYAEAVKG

SEQ ID NO: 16 - protein, 11 aa, synthetic construct
  REGION 1..11: note = CDR-H3 (Fremanezumab)
YFDYGLAIQNY

SEQ ID NO: 17 - protein, 122 aa, synthetic construct
  REGION 1..122: note = VH (Fremanezumab)
EVQLVESGGGLVQPGGSLRLSCAASGFTFSNYWISWVRQAPGKGLEWVAEIRSESDASATHYAEAVKGRFTISRDNAKNSLYLQMNSLRAEDTAVYYCLAYFDYGLAIQNYWGQGTLVTVSS

SEQ ID NO: 18 - protein, 448 aa, synthetic construct
  REGION 1..448: note = HC (Fremanezumab)
EVQLVESGGGLVQPGGSLRLSCAASGFTFSNYWISWVRQAPGKGLEWVAEIRSESDASATHYAEAVKGRFTISRDNAKNSLYLQMNSLRAEDTAVYYCLAYFDYGLAIQNYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 19 - protein, 11 aa, synthetic construct
  REGION 1..11: note = CDR-L1(Fremanezumab)
KASKRVTTYVS

SEQ ID NO: 20 - protein, 7 aa, synthetic construct
  REGION 1..7: note = CDR-L2 (Fremanezumab)
GASNRYL

SEQ ID NO: 21 - protein, 9 aa, synthetic construct
  REGION 1..9: note = CDR-L3 (Fremanezumab)
SQSYNYPYT

SEQ ID NO: 22 - protein, 107 aa, synthetic construct
  REGION 1..107: note = VL (Fremanezumab)
EIVLTQSPATLSLSPGERATLSCKASKRVTTYVSWYQQKPGQAPRLLIYGASNRYLGIPARFSGSGSGTDFTLTISSLEPEDFAVYYCSQSYNYPYTFGQGTKLEIK

SEQ ID NO: 23 - protein, 214 aa, synthetic construct
  REGION 1..214: note = LC (Fremanezumab)
EIVLTQSPATLSLSPGERATLSCKASKRVTTYVSWYQQKPGQAPRLLIYGASNRYLGIPARFSGSGSGTDFTLTISSLEPEDFAVYYCSQSYNYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 5 aa, synthetic construct
  REGION 1..5: note = CDR-H1 (Galcanezumab)
NYWMQ

SEQ ID NO: 25 - protein, 17 aa, synthetic construct
  REGION 1..17: note = CDR-H2 (Galcanezumab)
AIYEGTGKTVYIQKFAD

SEQ ID NO: 26 - protein, 10 aa, synthetic construct
  REGION 1..10: note = CDR-H3 (Galcanezumab)
LSDYVSGFGY

SEQ ID NO: 27 - protein, 119 aa, synthetic construct
  REGION 1..119: note = VH (Galcanezumab)
QVQLVQSGAEVKKPGSSVKVSCKASGYTFGNYWMQWVRQAPGQGLEWMGAIYEGTGKTVYIQKFADRVTITADKSTSTAYMELSSLRSEDTAVYYCARLSDYVSGFGYWGQGTTVTVSS

SEQ ID NO: 28 - protein, 445 aa, synthetic construct
  REGION 1..445: note = HC (Galcanezumab)
QVQLVQSGAEVKKPGSSVKVSCKASGYTFGNYWMQWVRQAPGQGLEWMGAIYEGTGKTVYIQKFADRVTITADKSTSTAYMELSSLRSEDTAVYYCARLSDYVSGFGYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 29 - protein, 11 aa, synthetic construct
  REGION 1..11: note = CDR-L1 (Galcanezumab)
RASKDISKYLN

SEQ ID NO: 30 - protein, 7 aa, synthetic construct
  REGION 1..7: note = CDR-L2 (Galcanezumab)
YTSGYHS

SEQ ID NO: 31 - protein, 9 aa, synthetic construct
  REGION 1..9: note = CDR-L3 (Galcanezumab)
QQGDALPPT

SEQ ID NO: 32 - protein, 107 aa, synthetic construct
  REGION 1..107: note = VL (Galcanezumab)
DIQMTQSPSSLSASVGDRVTITCRASKDISKYLNWYQQKPGKAPKLLIYYTSGYHSGVPSRFSGSGSGTDFTLTISSLQPEDFATYYCQQGDALPPTFGGGTKVEIK

SEQ ID NO: 33 - protein, 214 aa, synthetic construct
  REGION 1..214: note = LC (Galcanezumab)
DIQMTQSPSSLSASVGDRVTITCRASKDISKYLNWYQQKPGKAPKLLIYYTSGYHSGVPSRFSGSGSGTDFTLTISSLQPEDFATYYCQQGDALPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 5 aa, synthetic construct
  REGION 1..5: note = CDR-H1 (Erenumab)
SFGMH

SEQ ID NO: 35 - protein, 17 aa, synthetic construct
  REGION 1..17: note = CDR-H2 (Erenumab)
VISFDGSIKYSVDSVKG

SEQ ID NO: 36 - protein, 21 aa, synthetic construct
  REGION 1..21: note = CDR-H3 (Erenumab)
DRLNYYDSSGYYHYKYYGMAV

SEQ ID NO: 37 - protein, 130 aa, synthetic construct [sequence omitted]

SEQ ID NO: 38 - protein, 456 aa, synthetic construct
  REGION 1..456: note = HC (Erenumab)
QVQLVESGGGVVQPGRSLRLSCAASGFTFSSFGMHWVRQAPGKGLEWVAVISFDGSIKYSVDSVKGRFTISRDNSKNTLFLQMNSLRAEDTAVYYCARDRLNYYDSSGYYHYKYYGMAVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 39 - protein, 13 aa, synthetic construct
  REGION 1..13: note = CDR-L1 (Erenumab)
SGSSSNIGNNYVS

SEQ ID NO: 40 - protein, 7 aa, synthetic construct
  REGION 1..7: note = CDR-L2 (Erenumab)
DNNKRPS

SEQ ID NO: 41 - protein, 11 aa, synthetic construct
  REGION 1..11: note = CDR-L3 (Erenumab)
GTWDSRLSAVV

SEQ ID NO: 42 - protein, 110 aa, synthetic construct
  REGION 1..110: note = VL (Erenumab)
QSVLTQPPSVSAAPGQKVTISCSGSSSNIGNNYVSWYQQLPGTAPKLLIYDNNKRPSGIPDRFSGSKSGTSTTLGITGLQTGDEADYYCGTWDSRLSAVVFGGGTKLTVL

SEQ ID NO: 43 - protein, 216 aa, synthetic construct
  REGION 1..216: note = LC (Erenumab)
QSVLTQPPSVSAAPGQKVTISCSGSSSNIGNNYVSWYQQLPGTAPKLLIYDNNKRPSGIPDRFSGSKSGTSTTLGITGLQTGDEADYYCGTWDSRLSAVVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

(19) United States
(12) Patent Application Publication
(10) Pub. No.: US 2024/0352107 A1
(43) Pub. Date: Oct. 24, 2024

(54) ANTI-CGRP ANTIBODY DOSING AND SCREENING METHODS

(71) Applicant: H. LUNDBECK A/S, Valby (DK)

(72) Inventors: Roger K. CADY, Ozark, MO (US); Joseph HIRMAN, Mill Creek, WA (US); Carlton ANDERSON, Dallas, TX (US); Elizabeth BRUNNER, Chicago, IL (US)

(21) Appl. No.: 18/685,718

(22) PCT Filed: Sep. 15, 2022
(86) PCT No.: PCT/IB2022/058723

§ 371 Date: Feb. 22, 2024

Related U.S. Application Data
(60) Provisional application No. 63/244,466, filed on Sep. 15, 2021.

(51) Int. Cl. C07K 16/18; A61K 39/00; A61P 25/06

(52) U.S. Cl. CPC C07K 16/18; A61P 25/06; A61K 2039/505; A61K 2039/545; C07K 2317/24

(57) ABSTRACT

The present invention relates the use of monoclonal anti-calcitonin gene-related peptide (CGRP) antagonist antibodies or anti-CGRP receptor antibodies in subjects that do not respond adequately to a first dose but respond to the second dose.

### PRIOR APPLICATION INFORMATION

This application claims priority to U.S. Provisional Application No. 63/244,466 filed Sep. 15, 2021, the contents of which is herein incorporated by reference in its entirety.

### FIELD OF THE INVENTION

The present invention relates the use of monoclonal anti-calcitonin gene-related peptide (CGRP) antagonist antibodies or anti-CGRP receptor antibodies in subjects that do not respond adequately to a first dose.

### SEQUENCE DISCLOSURE

This application includes as part of its disclosure an electronic sequence listing text file named “1143257o009813_1270.xml”, having a size of 57,321 bytes and created on Sep. 7, 2022, which is hereby incorporated by reference in its entirety.

### SEQUENCES NOT PERMITTED TO BE ENTERED IN ST.26 XML FILE DUE TO SEQUENCE LENGTH

Table A below lists sequences present in the priority application U.S. Provisional Application No. 63/244,466 (identified above, which is herein incorporated by reference in its entirety), but cannot be included in the 1143257o009813_1270.xml file submitted herewith due to the length of the sequences.

TABLE A

| | | | | | | | | | | Previously |
|---|---|---|---|---|---|---|---|---|---|---|
| | | | | | Contains | Sequence | | | | Presented In |
| | | | | | DNA and | Skipped | | | | Priority |
| Item | | | Molecule | | RNA | in XML | | Feature | | Application |
| No. | Residues | Length | Type | Organism | fragments | file | Feature Key | Location | Qualifiers | As SEQ ID |
| 1 | GDI | 3 | AA | synthetic | No | Yes | REGION | 1 . . . 3 | note = | SEQ ID No. 5 |
| | | | | construct | | | source | 1 . . . 3 | CDR-H3 | |
| | | | | | | | | | Vyepti | |
| | | | | | | | | | mol_type = | |
| | | | | | | | | | protein | |
| | | | | | | | | | organism = | |
| | | | | | | | | | synthetic | |
| | | | | | | | | | construct | |

### BACKGROUND OF THE INVENTION

Migraine is a neurological condition characterized by attacks of headache and associated symptoms, such as nausea, vomiting, photophobia, and/or phonophobia. In US and Western Europe, the overall prevalence of migraine sufferers is 11% of the general population (6% males; 15-18% females). The two most common forms of migraine, migraine without aura and migraine with aura, occur on less than 15 days per month and are referred to as episodic forms of migraine (EM) (Lipton et al, Neurology 68(5):343-349, 2007). However, 3% to 6% of individuals with EM evolve, in any given year, to a significantly more disabling condition called chronic migraine (CM) (Scher et al, Pain 106(1-2):81-89, 2003). Individuals with CM present with headaches of any severity on 15 or more days per month and have full-blown migraine on at least 8 days per month. A sizable proportion of individuals with CM experience daily headaches and, therefore, face considerable disability (Bigal and Lipton, Neurology 71(11):848-855, 2008).

Preventive drug treatment of migraine may be appropriate in a number of instances, including where frequency of attacks per month is two or higher, or where a patient's quality of life is severely impaired (Evers et al., Europ. J. Neurol. 16:968-981, 2009). A number of drugs from different pharmacological categories (e.g. beta blockers, anticonvulsants) have been approved for migraine prevention or have class A evidence to support their use. However, patient response and tolerance to some of these medications varies, and compliance and adherence to these medications can be poor (Puledda et al., J. Neurol. March 20. doi: 10.1007/s00415-017-8434, 2017).

Calcitonin gene-related peptide (CGRP) is a neuropeptide that has been found to be involved in migraine processes, both centrally and peripherally (Eftekhari and Edvinsson, Ther. Adv. Neurol. Disord. 3(6):369-378, 2010, Olesen, Cephalagia 31(5):638, 2011). Jugular levels of CGRP are increased during migraine attacks, and intravenous (iv) CGRP administration induces migraine-like headache in most individuals with migraine (Ashina et al., Neurology 55(9):1335-1340, 2000, Hansen et al., Cephalagia 30(1):1179-1186, 2010). CGRP is involved in the pathophysiology of migraine at all levels, peripherally (vasodilation, inflammation, and protein extravasation), at the trigeminal ganglion, and inside the brain (Ho et al., Nat. Rev. Neurol. 6(10):573-582, 2010). Studies have shown that inhibition of CGRP or antagonizing CGRP receptor has demonstrated efficacy in the treatment of EM (Bigal et al., Lancet Neurol. 14:1081-1090, 2015a, Hewitt et al., Cephalagia 31(6):712-722, 2011, Ho et al., Lancet 372(9656):2115-2123, 2008, Olesen et al., N. Engl. J. Med. 350(11):1104-1110, 2004) and CM (Bigal et al., Lancet Neurol. 14:1091-1100, 2015b). Patients with frequent migraine and their treating healthcare providers face complex treatment decisions. Individuals with episodic or chronic migraine experience migraine-related disability and reduced quality of life, and these impacts worsen with increasing migraine frequency. Reducing the frequency of migraine attacks is a key goal of preventive treatment. Close to 40% of individuals with episodic migraine and all of those with chronic migraine are considered candidates for preventive migraine treatment (American Headache Society, Headache. 2019;59(1):1-18; Adams et al., Cephalalgia. 2015;35(7):563-578).

Eptinezumab is a monoclonal antibody used for the preventive treatment of migraine, administered every 12 weeks. Phase 3 clinical studies (“PROMISE-1” and “PROMISE-2” clinical studies) assessed the efficacy and safety of eptinezumab in patients with episodic and chronic migraine, respectively, administered with 12-week intervals between doses (Ashina et al. Cephalalgia. 2020;40(3):241-254; Lipton et al., Neurology 2020;94(13):e1365-e1377). In both studies, the first dose of eptinezumab led to reductions in mean monthly migraine days (MMDs) over weeks 1-12 (the primary study period; PROMISE-1: placebo, −3.2; eptinezumab 30 mg, −4.0; eptinezumab 100 mg, −3.9 [p=0.0182 vs placebo]; eptinezumab 300 mg, −4.3 [p=0.0001 vs placebo]. PROMISE-2: placebo: −5.6, eptinezumab 100 mg: −7.7 [p<0.0001 vs placebo]; eptinezumab 300 mg: −8.2 [p<0.0001 vs placebo]).

A 50% reduction in migraine days relative to baseline in controlled trials of preventive treatments is generally considered to be a positive response to treatment and a useful benchmark in both clinical trials and clinical practice. In the PROMISE-1 and PROMISE-2 studies, treatment response, defined as an at least 50% reduction in MMDs, occurred in about 50-60% of patients in the eptinezumab treatment arms over weeks 1-12 (PROMISE-1: 49.8% and 56.3% with eptinezumab 100 mg, and 300 mg, respectively, vs 37.4% with placebo [p=0.0085 for eptinezumab 100 mg vs placebo, not statistically significant per the testing hierarchy; p=0.0001 for eptinezumab 300 mg vs placebo]; PROMISE-2: 57.6% and 61.4% with eptinezumab 100 mg and 300 mg, respectively, vs 39.3% with placebo [both p<0.0001]) (Ashina et al., Cephalalgia. 2020;40(3):241-254; Lipton et al., Neurology 2020;94(13):e1365-e1377). Treatment response with eptinezumab was observed on day 1 after dosing and was sustained across the entire treatment period. In clinical practice, healthcare providers may want to know the likelihood of response with a second dose of eptinezumab for patients experiencing a less than 50% MMD response to a first dose of eptinezumab.

The inventors of the present invention have found using post hoc analysis of clinical trials (PROMISE-1 and PROMISE-2data) that individuals with migraine who do not experience a positive result to their first dose of eptinezumab (a reduction of MMD less than 50% (<50%) MMD) can still benefit from a second dose of eptinezumab. For example, for patients with migraine who do not respond and have no change in MMDs after their first eptinezumab dose, the likelihood of having a response in MMD above 50% to a second dose is approximately 20% (episodic migraine) and 5-10% (chronic migraine). For most patients Eptinezumab has a very quick action, e.g., within a day, due to particular characteristics of Eptinezumab (see e.g., WO2020/222892, e.g., FIGS. 47 and 50 and David L., et al. Neurology April 2021;96 (15 Supplement) 1477). This rapid action of Eptinezumab entails that if no treatment effect is observed following the first dose, that specific patient would likely be expected to be a suboptimal responder. However, surprisingly the inventors of the present invention have identified that if a treatment effect does not appear following a single dose, such treatment effect can still be observed in these suboptimal responders following a second dose of Eptinezumab.

### SUMMARY OF THE INVENTION

The present invention relates to a method of treating or preventing headache in a subject, wherein the method comprises:
i) selecting a subject who has been treated with a first dose of a humanized monoclonal anti-calcitonin gene-related peptide (CGRP) antagonist antibody or anti-CGRP receptor antibody, which subject has <50% reduction from baseline in Monthly Migraine Days (“MMD response”) after said first dose, and
ii) administering to the subject second dose of a therapeutically effective amount of a humanized monoclonal anti-calcitonin gene-related peptide (CGRP) antagonist antibody or anti-CGRP receptor antibody.

### DEFINITIONS

As used herein, the term “reduction in migraine” refers to a reduction (e.g., a stated percentage reduction, such as 50%) in the likelihood of a patient having a migraine in the stated period, such as the 18 hour, 20 hour, 24 hour, 28 hour, or 30 hour period, preferably the 24 hour period, after a first dosage of an antibody, or on the first day following the day of antibody administration (i.e., on the first full day following the day on which the antibody administration is completed). It is to be understood that a given patient may or may not have a migraine during that period, as the reduction in likelihood may be observable over a large number of patients irrespective of the outcome for an individual patient.

As used herein, the term “chronic migraine” refers to a condition wherein a patient exhibits, on average, at least 15 migraine days and/or headache per month.

As used herein, the term “episodic migraine” refers to a condition wherein a patient exhibits, on average, less than 15 headache and/or migraine days per month (IHS classification ICHD 3).

As used herein, the term “cluster headache” may also be defined as “episodic”, with cluster headache attacks occurring in periods lasting from 7 days to one year, separated by pain-free periods lasting at least 3 months, or “chronic” where the attacks are occurring for one year or longer without remission, of with remission periods lasting less than 3 months (IHS classification ICHD 3).

As used herein, the term “diagnosed with chronic migraine” refers to a patient meeting the clinical criteria for chronic migraine, whether or not a formal diagnosis of that patient was performed.

As used herein, the term “intravenously administering” refers to a mode of administration wherein a substance, e.g., an antibody, is introduced directly into the circulation of that patient. The substance may be introduced in a carrier fluid, such as an aqueous solution, e.g., normal saline. The substance may be administered in a single formulation or in multiple formulations, as long as the administration is completed over a short period of time (e.g., within 1 day, preferably within 12 hours, more preferably within 6 hours, and most preferably within 1-2 hours).

MMD means monthly migraine days.

As used herein, the term “the baseline number of MMD” refers to the number of MMD days exhibited by a patient prior to treatment with an anti-CGRP antagonist antibody or anti-CGRP receptor antibody or an anti-CGRP antagonist antibody fragment or anti-CGRP receptor antibody fragment such as Eptinezumab, Fremanezumab, Galcanezumab or Erenumab.

Suboptimal responders are defined herein as patients with a <50% (less than 50%) reduction from baseline in MMD, such as less than 45%, less than 35% or less 25%, less than 15%, less than 5% or not responding (0%); responders were defined as patients with a ≥50% (equal to or more than 50%) reduction from baseline in MMDs from baseline.

The Headache Impact Test-6 (HIT-6) was used to measure a wide spectrum of the factors contributing to the burden of headache, generating quantitative and pertinent information on the impact of headache The invention relates to methods of treating subjects (see e.g. Shin et al., J Clin Neurol. 2008 December; 4(4):158-163.).

Cluster headache may also be measured in monthly cluster headache days.

As used herein the term subject(s) is a human, in particular a headache patient.

The general structure of antibodies in vertebrates now is well understood (Edelman, G. M., Ann. N.Y. Acad. Sci., 190:5(1971)). Antibodies consist of two identical light polypeptide chains of molecular weight approximately 23,000 daltons (the “light chain”), and two identical heavy chains of molecular weight 53,000-70,000 (the “heavy chain”). The four chains are joined by disulfide bonds in a “Y” configuration wherein the light chains bracket the heavy chains starting at the mouth of the “Y” configuration. The “branch” portion of the “Y” configuration is designated the F_{ab} region; the stem portion of the “Y” configuration is designated the F_{C} region. The amino acid sequence orientation runs from the N-terminal end at the top of the “Y” configuration to the C-terminal end at the bottom of each chain. The N-terminal end possesses the variable region having specificity for the antigen that elicited it, and is approximately 100 amino acids in length, there being slight variations between light and heavy chain and from antibody to antibody.

The variable region is linked in each chain to a constant region that extends the remaining length of the chain and that within a particular class of antibody does not vary with the specificity of the antibody (i.e., the antigen eliciting it). There are five known major classes of constant regions that determine the class of the immunoglobulin molecule (IgG, IgM, IgA, IgD, and IgE corresponding to γ, μ, α, δ, and ε (gamma, mu, alpha, delta, or epsilon) heavy chain constant regions). The constant region or class determines subsequent effector function of the antibody, including activation of complement (Kabat, E. A., Structural Concepts in Immunology and Immunochemistry, 2nd Ed., p. 413-436, Holt, Rinehart, Winston (1976)), and other cellular responses (Andrews, D. W., et al., Clinical Immunobiology, pp 1-18, W. B. Sanders (1980); Kohl, S., et al., Immunology, 48:187 (1983)); while the variable region determines the antigen with which it will react. Light chains are classified as either κ (kappa) or λ (lambda). Each heavy chain class can be prepared with either kappa or lambda light chain. The light and heavy chains are covalently bonded to each other, and the “tail” portions of the two heavy chains are bonded to each other by covalent disulfide linkages when the immunoglobulins are generated either by hybridomas or by B cells.

The expression “variable region” or “VR” refers to the domains within each pair of light and heavy chains in an antibody that are involved directly in binding the antibody to the antigen. Each heavy chain has at one end a variable domain (V_{H}) followed by a number of constant domains. Each light chain has a variable domain (V_{L}) at one end and a constant domain at its other end; the constant domain of the light chain is aligned with the first constant domain of the heavy chain, and the light chain variable domain is aligned with the variable domain of the heavy chain.

The expressions “complementarity determining region,” “hypervariable region,” or “CDR” refer to one or more of the hyper-variable or complementarity determining regions (CDRs) found in the variable regions of light or heavy chains of an antibody (See Kabat, E. A. et al., Sequences of Proteins of Immunological Interest, National Institutes of Health, Bethesda, Md., (1987)). These expressions include the hypervariable regions as defined by Kabat et al. (“Sequences of Proteins of Immunological Interest,” Kabat E., et al., US Dept. of Health and Human Services, 1983) or the hypervariable loops in 3-dimensional structures of antibodies (Chothia and Lesk, J Mol. Biol. 196 901-917 (1987)). The CDRs in each chain are held in close proximity by framework regions and, with the CDRs from the other chain, contribute to the formation of the antigen binding site. Within the CDRs there are select amino acids that have been described as the selectivity determining regions (SDRs) which represent the critical contact residues used by the CDR in the antibody-antigen interaction (Kashmiri, S., Methods, 36:25-34 (2005)). In the present invention when specific antibody amino acid residues are referenced by number this generally refers to its position within a specified amino acid sequence (i.e., particular sequence identifier) and/or in accordance with Kabat et al numbering.

The expressions “framework region” or “FR” refer to one or more of the framework regions within the variable regions of the light and heavy chains of an antibody (See Kabat, E. A. et al., Sequences of Proteins of Immunological Interest, National Institutes of Health, Bethesda, Md., (1987)). These expressions include those amino acid sequence regions interposed between the CDRs within the variable regions of the light and heavy chains of an antibody.

Calcitonin Gene Related Peptide (CGRP): As used herein, CGRP encompasses not only the following Homo sapiens CGRP-alpha and Homo sapiens CGRP-beta amino acid sequences available from American Peptides (Sunnyvale CA) and Bachem (Torrance, CA):

CGRP-alpha: ACDTATCVTHRLAGLLSRSGGVVKNNFVPTNVGSKAF-NH₂, wherein the terminal phenylalanine is amidated (SEQ ID No.: 1)

CGRP-beta: ACNTATCVTHRLAGLLSRSGGMVKSNFVPTNVGSKAF-NH₂, wherein the terminal phenylalanine is amidated (SEQ ID No.: 2); but also any membrane-bound forms of these CGRP amino acid sequences, as well as mutants (mutiens), splice variants, isoforms, orthologs, homologues and variants of this sequence.

### BRIEF DESCRIPTION OF FIGURES

FIG. 1. Potential first-infusion predictors of second-infusion ≥50% MMD response in patients who had a <50% MMD response across weeks 1-12, using full logistic regression analysis (eptinezumab 100 mg and 300 mg treatment groups, pooled data).

FIG. 2. Stepwise logistic regression analysis of first-infusion predictors of second-infusion ≥50% MMD response in patients who had a <50% MMD response across weeks 1-12 (eptinezumab 100 mg and 300 mg treatment groups, pooled data).

FIG. 3. Probability of second-infusion ≥50% MMD response based on first-infusion predictors in the PROMISE-1 study.

FIG. 4. Probability of second-infusion ≥50% MMD response based on first-infusion predictors in the PROMISE-2 study.

FIG. 5A-B. Probability of second-infusion ≥50% MMD response based on first-infusion predictors in (A) the PROMISE-1 study and (B) the PROMISE-2 study.

### DETAILED DESCRIPTION OF THE INVENTION

Phase III clinical studies (PROMISE-1 and PROMISE-2 clinical studies) assessed the effects of eptinezumab treatment for patients with episodic and chronic migraine. A key secondary endpoint in both studies was the proportion of patients who had a ≥50% reduction in MMDs over weeks 1-12. Approximately 50-60% of patients in the PROMISE-1 and PROMISE-2 studies responded to the first dose of eptinezumab with a ≥50% MMD reduction. Healthcare providers and their patients in everyday clinical practice may want to know the likelihood of response with a second dose of eptinezumab for patients with initial suboptimal to a first dose of eptinezumab. In this post hoc analysis of pooled eptinezumab 100 mg and 300 mg dose data from the PROMISE-1 and PROMISE-2 studies, among patients who did not respond with a ≥50% MMD reduction to their first eptinezumab infusion, about one-third (37.0% of those with episodic migraine and 28.8% of those with chronic migraine) were ≥50% MMD responders to their second eptinezumab dose. Percent change in MMDs was demonstrated to be a significant first dose predictor of second dose response for individuals with episodic migraine, and percent change in MMDs and change in HIT-6 total score (assessed in PROMISE-2 only) were shown to be significant first dose predictors of second dose response for patients with chronic migraine.

For patients with episodic migraine who experience no change in MMDs in response to the first dose, the probability of having a ≥50% MMD response to a second dose was approximately 20%. Hence, even patients with no change in MMD after their first dose have a one-in-five probability of responding to a second dose of eptinezumab. The probability of being a ≥50% MMD responder to the second dose increases to about 60% as a patient's percent change in MMDs after the first dose nears 50%. For patients with chronic migraine who experience no change in MMDs in response to the first dose, depending on change in HIT-6 total score, the probability of having a ≥50% MMD response to a second dose was approximately 5-10% and increased to about 60-80% as a patient's percent change in MMDs after the first dose nears 50%.

According to one aspect the present invention relates to a method of treating or preventing headache in a subject the method comprises:
i) selecting a subject who has been treated with a humanized monoclonal anti-calcitonin gene-related peptide (CGRP) antagonist antibody, which subject has <50% reduction from baseline in Monthly Migraine Days (“MMD response”) after first dose, and
ii) administering to the subject second dose of said humanized anti-CGRP antagonist antibody, which second dose comprises a therapeutically effective amount of said humanized monoclonal anti-CGRP antagonist antibody,
   wherein said humanized monoclonal anti-CGRP antagonist antibody comprises a heavy chain variable region comprising the amino acid sequence set forth in SEQ ID NO: 6 and a light chain variable region comprising the amino acid sequence set forth in SEQ ID NO: 12.

In certain embodiments the subject has less than 45%, less than 35% or less 25%, less than 15%, less than 5% or not responding (0%) MMD response after first dose.

According to some embodiments the subject may have migraine or cluster headache, such as episodic migraine, chronic migraine, episodic cluster headache or chronic cluster headache. The subject may experience migraines with aura or migraines without aura.

The subject is a human according to one embodiment or a human headache patient.

The subject in step i) above has only received one dosage of said humanized monoclonal anti-calcitonin gene-related peptide (CGRP) antagonist antibody, and may according to one embodiment receive the same dosage in step i) and step ii) or a dosage that differs in step i) and step ii). The dose or doses may be given subcutaneously or intravenously.

According to one embodiment the monoclonal antibody is administered at a dose of about 100 mg to about 500 mg in step i) and at a dose of about 100 mg to about 500 mg in step ii).

According to another embodiment the dose is about 100 mg, about 300 mg or about 400 mg in step i) and is about 100 mg, about 300 mg or about 400 mg in step ii).

In some embodiments the monoclonal antibody is administered once per 3 months (about 12 weeks).

According to one embodiment the subject in step i) does not experience a ≥50% MMD response to their first dose of said humanized monoclonal anti-calcitonin gene-related peptide (CGRP) antagonist antibody which is indicative of a suboptimal responder.

The subject may according to other embodiments not experience a ≥50% MMD response to their first dose of said humanized monoclonal anti-calcitonin gene-related peptide (CGRP) antagonist antibody within 1 to 12 weeks, within 1 to 4 weeks, or within 1 to 2 weeks.

In some aspects, said migraine or headache may be selected from the group comprising acute migraine or headache, migraines with or without aura, chronic migraine, episodic migraine, chronic/episodic migraine, hemiplegic migraines, cluster headaches, migrainous neuralgia, chronic headaches, tension headaches, general headaches, headaches due to an underlying structural problem in the head or neck, sinus headaches (such as for example associated with sinusitis), and allergy-induced headaches or migraines.

The antibody according to the invention (Vyepti, or Eptinezumab) comprises the following CDRs as determined by Kabat (Kabat, E. A., Structural Concepts in Immunology and Immunochemistry, 2nd Ed., p. 413-436)

Heavy Chain CDRs for Eptinezumab
CDR-H1:
SEQ ID No.: 3
GYYMN
CDR-H2:
SEQ ID No.: 4
VIGINGATYYASWAKG
CDR-H3: GDI RESIDUE GDI (SEE PAGE 1, TABLE A, ITEM NO. 1)
The Variable Heavy Chain of Eptinezumab comprises:
SEQ ID No.: 6
EVQLVESGGGLVQPGGSLRLSCAVSGIDLSGYYMNWVRQAPGKGLEWVGVIGINGATYYASWAKG
RFTISRDNSKTTVYLQMNSLRAEDTAVYFCARGDIWGQGTLVTVSS
The Heavy Chain of Eptinezumab comprises:
SEQ ID No.: 7
EVQLVESGGG LVQPGGSLRL SCAVSGIDLS GYYMNWVRQA PGKGLEWVGV
IGINGATYYA SWAKGRFTIS RDNSKTTVYL QMNSLRAEDT AVYFCARGDI
WGQGTLVTVS SASTKGPSVF PLAPSSKSTS GGTAALGCLV KDYFPEPVTV
SWNSGALTSG VHTFPAVLQS SGLYSLSSVV TVPSSSLGTQ TYICNVNHKP
SNTKVDARVE PKSCDKTHTC PPCPAPELLG GPSVFLFPPK PKDTLMISRT
PEVTCVVVDV SHEDPEVKFN WYVDGVEVHN AKTKPREEQY ASTYRVVSVL
TVLHQDWLNG KEYKCKVSNK ALPAPIEKTI SKAKGQPREP QVYTLPPSRE
EMTKNQVSLT CLVKGFYPSD IAVEWESNGQ PENNYKTTPP VLDSDGSFFL
YSKLTVDKSR WQQGNVFSCS VMHEALHNHY TQKSLSLSPG

A C-terminal lysine (K) may be present in case the Heavy Chain is not processed or expressed in a system or cell that cleave this, e.g. in a yeast such as Pichia, but will usually be cleaved in a CHO expression system

The Heavy Chain of Eptinezumab will then comprise:
SEQ ID No.: 8
EVQLVESGGG LVQPGGSLRL SCAVSGIDLS GYYMNWVRQA PGKGLEWVGV
IGINGATYYA SWAKGRFTIS RDNSKTTVYL QMNSLRAEDT AVYFCARGDI
WGQGTLVTVS SASTKGPSVF PLAPSSKSTS GGTAALGCLV KDYFPEPVTV
SWNSGALTSG VHTFPAVLQS SGLYSLSSVV TVPSSSLGTQ TYICNVNHKP
SNTKVDARVE PKSCDKTHTC PPCPAPELLG GPSVFLFPPK PKDTLMISRT
PEVTCVVVDV SHEDPEVKFN WYVDGVEVHN AKTKPREEQY ASTYRVVSVL
TVLHQDWLNG KEYKCKVSNK ALPAPIEKTI SKAKGQPREP QVYTLPPSRE
EMTKNQVSLT CLVKGFYPSD IAVEWESNGQ PENNYKTTPP VLDSDGSFFL
YSKLTVDKSR WQQGNVFSCS VMHEALHNHY TQKSLSLSPGK
Light Chain CDRs for Eptinezumab
CDR-L1:
SEQ ID No.: 9
QASQSVYHNTYLA
CDR-L2:
SEQ ID No.: 10
DASTLAS
CDR-L3:
SEQ ID No.: 11
LGSYDCTNGDCFV
The Variable Light Chain of Eptinezumab comprises:
SEQ ID No.: 12
QVLTQSPSSLSASVGDRVTINCQASQSVYHNTYLAWYQQKPGKVPKQLIYDASTLASGVPSRFSGSG
SGTDFTLTISSLQPEDVATYYCLGSYDCTNGDCFVFGGGTKVEIKR
The Light Chain of Eptinezumab comprises:
SEQ ID No.: 13
QVLTQSPSSL SASVGDRVTI NCQASQSVYH NTYLAWYQQK PGKVPKQLIY
DASTLASGVP SRFSGSGSGT DFTLTISSLQ PEDVATYYCL GSYDCTNGDC
FVFGGGTKVE IKRTVAAPSV FIFPPSDEQL KSGTASVVCL LNNFYPREAK
VQWKVDNALQ SGNSQESVTE QDSKDSTYSL SSTLTLSKAD YEKHKVYACE
VTHQGLSSPV TKSFNRGEC

In some aspects, Vyepti (Eptinezumab) may be comprised in a formulation comprising or consisting of histidine (L-histidine), sorbitol, polysorbate 80, and water, such as, per 1 mL volume, about 100 mg or 300 mg Vyepti (Eptinezumab), about 3.1 mg L-Histidine, about 40.5 mg Sorbitol, and about 0.15 mg Polysorbate 80, having a pH of about 5.8.

In some aspects, said formulation may comprise or may consist of, per 1 mL volume, 100 or 300 mg Vyepti (Eptinezumab), 3.1 mg L-Histidine, 40.5 mg Sorbitol, and 0.15 mg Polysorbate 80, or having amounts of each constituent within +/−10% of said values, and having a pH of 5.8 or within +/−10% of said value. In some aspects, said formulation may comprise or may consist of, per 1 mL volume, 100 or 300 mg Vyepti (Eptinezumab), 3.1 mg L-Histidine, 40.5 mg Sorbitol, and 0.15 mg Polysorbate 80, or having amounts of each constituent within +/−5% of said values, and/or having a pH of 5.8 or within +/−5% of said value. In some aspects, said formulation may comprise or may consist of, per 1 mL volume, 100 mg Vyepti (Eptinezumab), 3.1 mg L-Histidine, 40.5 mg Sorbitol, and 0.15 mg Polysorbate 80, or having amounts of each constituent within +/−1% of said values, and/or having a pH of 5.8 or within +/−1% of said value. In some aspects, said formulation may comprise or may consist of, per 1 mL volume, 100 mg Vyepti (Eptinezumab), 3.1 mg L-Histidine, 40.5 mg Sorbitol, and 0.15 mg Polysorbate 80, or having amounts of each constituent within +/−0.5% of said values, and/or having a pH of 5.8 or within +/−0.5% of said value. In some aspects, said formulation may comprise or may consist of, per 1 mL volume, 100 mg Vyepti (Eptinezumab), 3.1 mg L-Histidine, 40.5 mg Sorbitol, and 0.15 mg Polysorbate 80, or having amounts of each constituent within +/−0.1% of said values, and/or having a pH of 5.8 or within +/−0.1% of said value. In some aspects, said L-Histidine in said formulation comprises a mixture of L-Histidine and L-Histidine monohydrate. Said 3.1 mg of histidine in said formulation may comprise a mixture of L-Histidine (1 mg) and L-Histidine monohydrate (2.8 mg), which in the final formulation sums up to 3.1 mg L-histidine free base. In some aspects, said formulation may be comprised in a 100 mg/mL single-dose vial wherein each mL contains 100 mg Vyepti (Eptinezumab), L-histidine (1 mg), L-histidine hydrochloride monohydrate (2.8 mg), polysorbate 80 (0.15 mg), sorbitol (40.5 mg), and Water for Injection, USP, at a pH of 5.8.

In some aspects, said formulation may be comprised in a 300 mg/mL single-dose vial wherein each mL contains 300 mg Vyepti (Eptinezumab), L-histidine (1 mg), L-histidine hydrochloride monohydrate (2.8 mg), polysorbate 80 (0.15 mg), sorbitol (40.5 mg), and Water for Injection, USP, at a pH of 5.8.

Vyepti (Eptinezumab) is preferably administered intravenously or subcutaneously.

According to another embodiment the invention also relates to other CGRP binding antibodies, for example, Fremanezumab or Galcanezumab.

Fremanezumab has the following sequences:

Fremanezumab Heavy Chain CDRs
CDR1-H1
SEQ ID No.: 14
NYWIS
CDR2-H2
SEQ ID No.: 15
EIRSESDASATHYAEAVKG
CDR3-H3
SEQ ID No.: 16
YFDYGLAIQNY
Heavy chain variable region
SEQ ID No.: 17
EVQLVESGGGLVQPGGSLRLSCAASGFTFSNYWISWVRQAPGKGLEWVAEIRSES
DASATHYAEAVKGRFTISRDNAKNSLYLQMNSLRAEDTAVYYCLAYFDYGLAIQNY WGQGTLVTVSS
Fremanezumab Heavy Chain region
SEQ ID No.: 18
EVQLVESGGGLVQPGGSLRLSCAASGFTFSNYWISWVRQAPGKGLEWVAEIRSES
DASATHYAEAVKGRFTISRDNAKNSLYLQMNSLRAEDTAVYYCLAYFDYGLAIQNY
WGQGTLVTVSSASTKGPSVFPLAPCSRSTSESTAALGCLVKDYFPEPVTVSWNSG
ALTSGVHTFPAVLQSSGLYSLSSVVTVPSSNFGTQTYTONVDHKPSNTKVDKTVER
KCCVECPPCPAPPVAGPSVFLFPPKPKDTLMISRTPEVTCVVVDVSHEDPEVQFNW
YVDGVEVHNAKTKPREEQFNSTFRVVSVLTVVHQDWLNGKEYKCKVSNKGLPSSI
EKTISKTKGQPREPQVYTLPPSREEMTKNQVSLTCLVKGFYPSDIAVEWESNGQPE
NNYKTTPPMLDSDGSFFLYSKLTVDKSRWQQGNVFSCSVMHEALHNHYTQKSLSL SPGK
Fremanezumab Light Chain CDRs
CDR1-L1
SEQ ID No.: 19
KASKRVTTYVS
CDR2-L2
SEQ ID No.: 20
GASNRYL
CDR3-L3
SEQ ID No.: 21
SQSYNYPYT
Fremanezumab Light chain variable region amino acid sequence
SEQ ID No.: 22
EIVLTQSPATLSLSPGERATLSCKASKRVTTYVSWYQQKPGQAPRLLIYGASNRYL
GIPARFSGSGSGTDFTLTISSLEPEDFAVYYCSQSYNYPYTFGQGTKLEIK
Fremanezumab Light Chain region
SEQ ID No.: 23
EIVLTQSPATLSLSPGERATLSCKASKRVTTYVSWYQQKPGQAPRLLIYGASNRYL
GIPARFSGSGSGTDFTLTISSLEPEDFAVYYCSQSYNYPYTFGQGTKLEIKRTVAAP
SVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGEC

The dosages given of Fremanezumab is 225 mg monthly or alternatively 675 mg quarterly (about 12 weeks), both by subcutaneous administrations.

According to one embodiment Fremanezumab is administered at a dose of about 200 mg to about 700 mg in step i) and a dose of about 200 mg to about 700 mg in step ii).

According to another embodiment the dose is about 225 mg or about 675 mg in step i) and about 225 mg or about 675 mg in step ii).

According to another embodiment the dose is about 225 mg is given monthly and the about 675 mg dosage is given quarterly (about 12 weeks) in step ii).

Galcanezumab has the following sequences:

Galcanezumab heavy chain CDRs
SEQ ID No.: 24
CDR1-H1
NYWMQ
CDR2-H2
SEQ ID No.: 25
AIYEGTGKTVYIQKFAD
CDR3-H3
SEQ ID No.: 26
LSDYVSGFGY
Galcanezumab Heavy chain variable region
SEQ ID No.: 27
QVQLVQSGAEVKKPGSSVKVSCKASGYTFGNYWMQWVRQAPGQGLEWMGAIYEGTGKTVYIQKF
ADRVTITADKSTSTAYMELSSLRSEDTAVYYCARLSDYVSGFGYWGQGTTVTVSS
Galcanezumab Heavy chain
SEQ ID No.: 28
QVQLVQSGAEVKKPGSSVKVSCKASGYTFGNYWMQWVRQAPGQGLEWMGAIYEGTGKTVY
IQKFADRVTITADKSTSTAYMELSSLRSEDTAVYYCARLSDYVSGFGYWGQGTTVTVSSA
STKGPSVFPLAPCSRSTSESTAALGCLVKDYFPEPVTVSWNSGALTSGVHTFPAVLQSSG
LYSLSSVVTVPSSSLGTKTYTCNVDHKPSNTKVDKRVESKYGPPCPPCPAPEAAGGPSVF
LFPPKPKDTLMISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTLPPSQEEMTKN
QVSLTCLVKGFYPSDIAVEWESNGQPENNYKTTPPVLDSDGSFFLYSRLTVDKSRWQEGN
VFSCSVMHEALHNHYTQKSLSLSLG
Galcanezumab light chain CDRs
CDR1-L1
SEQ ID No.: 29
RASKDISKYLN
CDR2-L1
SEQ ID No.: 30
YTSGYHS
CDR3-L3
SEQ ID No.: 31
QQGDALPPT
Galcanezumab Light chain variable region
SEQ ID No.: 32
DIQMTQSPSSLSASVGDRVTITCRASKDISKYLNWYQQKPGKAPKLLIYYTSGYHSGVPSRFSGSGS
GTDFTLTISSLOPEDFATYYCQQGDALPPTFGGGTKVEIK
Galcanezumab Light chain
SEQ ID No.: 33
DIQMTQSPSSLSASVGDRVTITCRASKDISKYLNWYQQKPGKAPKLLIYYTSGYHSGVPS
RFSGSGSGTDFTLTISSLQPEDFATYYCQQGDALPPTFGGGTKVEIKRTVAAPSVFIFPP
SDEQLKSGTASVVCLLNNFYPREAKVQWKVDNALQSGNSQESVTEQDSKDSTYSLSSTLT
LSKADYEKHKVYACEVTHQGLSSPVTKSFNRGEC

The dosages given of Galcanezumab generally is 120 mg monthly for migraine and 300 mg for Cluster headache, both by subcutaneous administration. The first dosage may be a loading dosage of about 240 mg.

According to one embodiment Galcanezumab is administered at a dose of about 120 mg to about 300 mg in step i) and a dose of about 120 mg to about 300 mg in step ii).

According to another embodiment the dose is about 120 mg, 240 mg or about 300 mg in step i) and about 120 mg or about 300 mg in step ii).

In some embodiments, an anti-CGRP receptor antibody such as Erenumab can be used in any of the methods described herein.

Erenumab comprises the following sequences:

Erenumab heavy chain CDRs
CDR1-H1
SEQ ID No.: 33
SFGMH
CDR2-H2
SEQ ID No.: 34
VISFDGSIKYSVDSVKG
CDR3-H3
SEQ ID No.: 35
DRLN YYDSSGYYHYKYYGM AV
Erenumab Heavy chain variable region
SEQ ID No.: 36
QVQLVESGGGVVQPGRSLRLSCAASGFTFSSFGMHWVRQAPGKGLEWVAVISFD
GSIKYSVDSVKGRFTISRDNSKNTLFLQMNSLRAEDTAVYYCARDRLNYYDSSGYY
HYKYYGMAVWGQGTTVTVSS
Erenumab Heavy chain
SEQ ID No.: 37
QVQLVESGGGVVQPGRSLRLSCAASGFTFSSFGMHWVRQAPGKGLEWVAVISFDGSIKYS
VDSVKGRFTISRDNSKNTLFLQMNSLRAEDTAVYYCARDRLNYYDSSGYYHYKYYGMAVW
GQGTTVTVSSASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVERKCCVECPPCP
APPVAGPSVFLFPPKPKDTLMISRTPEVTCVVVDVSHEDPEVQFNWYVDGVEVHNAKTKP
REEQFNSTFRVVSVLTVVHQDWLNGKEYKCKVSNKGLPAPIEKTISKTKGQPREPQVYTL
PPSREEMTKNQVSLTCLVKGFYPSDIAVEWESNGQPENNYKTTPPMLDSDGSFFLYSKLT
VDKSRWQQGNVFSCSVMHEALHNHYTQKSLSLSPGK
Erenumab light chain CDRs
CDR1-L1
SEQ ID No.: 39
SGSSSNIGNNYVS
CDR2-L2
SEQ ID No.: 40
DNNKRPS
CDR3-L3
SEQ ID No.: 41
GTWDSRLSAVV
Erenumab Light chain variable region
SEQ ID No.: 42
QSVLTQPPSVSAAPGQKVTISCSGSSSNIGNNYVSWYQQLPGTAPKLLIYDNNKRP
SGIPDRFSGSKSGTSTTLGITGLQTGDEADYYCGTWDSRLSAVVFGGGTKLTVL
Erenumab Light chain region
SEQ ID No.: 43
QSVLTQPPSVSAAPGQKVTISCSGSSSNIGNNYVSWYQQLPGTAPKLLIYDNNKRPSGIP
DRFSGSKSGTSTTLGITGLQTGDEADYYCGTWDSRLSAVVFGGGTKLTVLGQPKANPTVT
LFPPSSEELQANKATLVCLISDFYPGAVTVAWKADGSPVKAGVETTKPSKQSNNKYAASS
YLSLTPEQWKSHRSYSCQVTHEGSTVEKTVAPTECS

The dosages of Erenumab given generally are mg monthly or alternatively 140 mg monthly, both by subcutaneous administration.

According to one embodiment Erenumab is administered at a dose of about 50 mg to about 150 mg in step i) and a dose of about 50 mg to about 150 mg in step ii).

According to another embodiment the dose is about 70 mg or about 140 mg in step i) and about 70 mg or about 140 mg in step ii).

In some aspect, the humanized monoclonal anti-calcitonin gene-related peptide (CGRP) antagonist antibody or anti-CGRP receptor antibody of the invention may be formulated at a concentration of 400 mg/mL, such as 300 mg/mL concentration, a 150 mg/mL concentration or a 100 mg/mL concentration.

1.5 mL of solution containing 225 mg fremanezumab may be formulated in disodium ethylenediaminetetraacetic acid dihydrate (EDTA) (0.204 mg), L-histidine (0.815 mg), L-histidine hydrochloride monohydrate (3.93 mg), polysorbate-80 (0.3 mg), sucrose (99 mg), and Water for Injection, and has a pH of 5.5. According to an embodiment of the invention fremanezumab (at a concentration of 200-300 mg, e.g. 225 mg) may formulated in a composition comprising ethylenediaminetetraacetic acid dihydrate (EDTA), L-histidine, L-histidine hydrochloride monohydrate, polysorbate-80, sucrose (99 mg) and Water.

1 mL solution of 70 mg erenumab may be formulated with, acetate (1.5 mg), polysorbate 80 (0.10 mg), and sucrose (73 mg), pH of 5.2. According to an embodiment about 50-150 mg (such as 70 mg) erenumab may be formulated in acetate, polysorbate 80, and sucrose, pH about 5.

1 mL 120 mg galcanezumab may be formulated with L-histidine, (0.5 mg); L-histidine hydrochloride monohydrate (1.5 mg); Polysorbate 80, (0.5 mg); Sodium Chloride, (8.8 mg); Water for Injection. The pH range is 5.3-6.3. According to an embodiment about 120 mg galcanezumab may be formulated with L-histidine, L-histidine hydrochloride monohydrate; Polysorbate 80; Sodium Chloride; Water for Injection at a pH range of about 5.3 to about 6.3.

According to some aspect of the invention the subject may receive concomitant or additional treatment for pain or migraine, such as a triptan, an analgesic such as non-opioids or opioids/narcotics, acetaminophen, an NSAID, a combination medication, an ergotamine, or an ergot derivative. According to one embodiment said non-opioid analgesic comprises paracetamol (acetaminophen), acetylsalicylic acid (aspirin), another NSAID, or another non-opioid analgesic; said triptan comprises use of one or more of sumatriptan, zolmitriptan, naratriptan, rizatriptan, eletriptan, almotriptan, or frovatriptan; said opioid comprises use of one or more of oxycodone, tramadol, butorphanol, morphine, codeine, and hydrocodone; said combination medication comprises two drugs with analgesic effects (for example, paracetamol and codeine), an analgesic and an adjuvant (for example, paracetamol and caffeine) and/or said combination-analgesics comprises at least one opioid (such as tramadol, butorphanol, morphine, codeine, hydrocodone, or any combination thereof), barbiturate such as butalbital, and/or caffeine, and/or said combination-analgesic comprises acetylsalicylic acid (aspirin), paracetamol and caffeine. IV anesthetic such as lidocaine are also used sometimes as are occipital nerve blocks and sphenopalatine blocks with lidocaine or other similar compounds.

According to another aspect of the invention the invention also provides a method of treating or preventing migraine or cluster headache in a subject having refractory migraine or cluster headache, the method comprising:
(i) selecting a subject who has been treated with two or more different classes of preventative migraine treatments wherein at least one of the classes of preventative migraine treatments is selected from the group consisting of beta-blockers, anticonvulsants, tricyclics, calcium channel blockers, angiotensin II receptor antagonists, onabotulinumtoxinA, and valproates prior to step i) of the invention; and
(ii) administering to the subject a therapeutically effective amount of a humanized monoclonal anti-calcitonin gene-related peptide (CGRP) antagonist antibody of the invention or anti-CGRP receptor antibody of the invention,
(iii) selecting subject(s) who has/have <50% MMD response after first dose of the humanized monoclonal anti-calcitonin gene-related peptide (CGRP) antagonist antibody of the invention or anti-CGRP receptor antibody of the invention, and
(iv) administering to the subject second dose of a therapeutically effective amount of a humanized monoclonal anti-calcitonin gene-related peptide (CGRP) antagonist antibody or anti-CGRP receptor antibody of the invention.

In another embodiment said preventive migraine treatment may be selected from the group comprising two or more different preventative migraine treatments wherein at least one of the preventative migraine treatments is selected from topiramate, carbamazepine, divalproex sodium, sodium valproate, valproic acid, divalproex, flunarizine, candesartan, pizotifen, amitriptyline, venlafaxine, nortriptyline, duloxetine, atenolol, nadolol, metoprolol, propranolol, bisopropol, timolol, onabotulinumtoxin A, lisinopril, and oxeterone.

### EXEMPLARY EMBODIMENTS OF THE INVENTION

Different exemplary embodiments of the invention are set forth below.

### 1. Embodiments

1.1 A method of treating or preventing headache in a subject the method comprises:
i) selecting a subject who has been treated with a first dose of a humanized monoclonal anti-calcitonin gene-related peptide (CGRP) antagonist antibody or anti-CGRP receptor antibody, which subject has a <50% reduction from baseline in Monthly Migraine Day (MMD) response (“MMD response”) after said first dose, and
ii) administering to the subject a second dose of a therapeutically effective amount of a humanized monoclonal anti-calcitonin gene-related peptide (CGRP) antagonist antibody or anti-CGRP receptor antibody.

1.2 The method according to Embodiment 1.1, wherein after the first dose of said CGRP antagonist antibody or anti-CGRP receptor antagonist antibody the subject has a less than 45%, less than 35%, less than 25%, less than 15%, less than 5% or not responding (≈0%) MMD response.

1.3 The method according to Embodiment 1.1 or 1.2, wherein the subject has migraine or cluster headache.

1.4 The method according to Embodiment 1.1, 1.2 or 1.3, wherein said migraine is episodic migraine, chronic migraine, episodic cluster headache or chronic cluster headache.

1.5 The method according to Embodiment 1.3 or 1.4, wherein the subject experiences migraines with aura.

1.6 The method according to Embodiments 1.3 or 1.4, wherein the subject experiences migraines without aura.

1.7 The method according to any one of the preceding Embodiments, wherein the subject in step i) of Embodiment 1 has only received one dosage of said humanized monoclonal anti-calcitonin gene-related peptide (CGRP) antagonist antibody or anti-CGRP receptor antibody.

1.8 The method according to any one of the preceding Embodiments, wherein said subject received the same dosage in step i) and step ii) or a different dosage in step i) and step ii).

1.9 The method according to any one of the preceding Embodiments, wherein the monoclonal antibody is administered once per month or every 12 weeks.

1.10 The method according to any one of the preceding Embodiments, wherein said selected subject did not experience a ≥50% MMD response to their first dose of said humanized monoclonal anti-calcitonin gene-related peptide (CGRP) antagonist antibody or anti-CGRP receptor antibody.

1.11 The method according to any one of the preceding Embodiments, wherein said selected subject did not experience a ≥50% MMD response to their first dose of said humanized monoclonal anti-calcitonin gene-related peptide (CGRP) antagonist antibody or anti-CGRP receptor antibody within 1 to 12 weeks.

1.12 The method according to any one of the preceding Embodiments, wherein said selected subject did not experience a ≥50% MMD response to their first dose of said humanized monoclonal anti-calcitonin gene-related peptide (CGRP) antagonist antibody or anti-CGRP receptor antibody within 1 to 4 weeks.

1.13 The method according to any one of the preceding Embodiments, wherein said selected subject did not experience a ≥50% MMD response to their first dose of said humanized monoclonal anti-calcitonin gene-related peptide (CGRP) antagonist antibody or anti-CGRP receptor antibody within 1 to 2 weeks.

1.14 The method according to any one of the preceding Embodiments, wherein said humanized monoclonal anti-calcitonin gene-related peptide (CGRP) antagonist antibody or anti-CGRP receptor antibody comprises a heavy chain and/or variable heavy chain polypeptide that has at least 90%, or 95% or greater sequence homology to the heavy chain or variable heavy chain of Erenumab, Vyepti, Galcanezumab or Fremanezumab as disclosed herein.

1.15 The method according to any one of the preceding Embodiments, wherein said humanized monoclonal anti-calcitonin gene-related peptide (CGRP) antagonist antibody or anti-CGRP receptor antibody comprises a light chain and/or variable light chain polypeptide that has at least 90%, or 95% or greater sequence homology to the light chain and/or variable light chain of Erenumab, Vyepti, Galcanezumab or Fremanezumab as disclosed herein.

1.16 The method according to any one of the preceding Embodiments, wherein said humanized monoclonal anti-calcitonin gene-related peptide (CGRP) antagonist antibody or anti-CGRP receptor antibody comprises a heavy chain and/or variable heavy chain polypeptide and a light chain and/or variable light chain polypeptide that each have at least 90%, or 95% or greater sequence homology to the heavy chain and/or variable heavy chain polypeptide and the light chain and/or variable light chain polypeptide of one of Erenumab, Vyepti, Galcanezumab or Fremanezumab as disclosed herein.

1.17 The method according to any one of the preceding Embodiments, wherein the CGRP or CGRP receptor antibody is Fremanezumab and the dosage of Fremanezumab is about 200 mg to about 700 mg in step i) and a dose of about 200 mg to about 700 mg is administered in step ii) of Embodiment 1, such as e.g. about 225 mg or about 675 mg in step i) and is about 225 mg or about 675 mg in step ii) preferably wherein the about 225 mg dosage is given monthly and the about 675 mg dosage is given quarterly in step i) and ii).

1.18 The method according to any one of the preceding Embodiments, wherein said the antibody is Galcanezumab and the dosage of Galcanezumab is about 120 mg to about 300 mg in step i) and a dose of about 120 mg to about 300 mg in step ii) of Embodiment 1, or the dose is about 120 mg, 240 mg or about 300 mg in step i) and is about 120 mg or about 300 mg in step ii), wherein said dose(s) is/are preferably given monthly.

1.19 The method according to any one of the preceding Embodiments, wherein said dosage of Erenumab is about 50 mg to about 150 mg in step i) and a dose of about 50 mg to about 150 mg in step ii) of Embodiment 1 or the dose is about 70 mg or about 140 mg in step i) and about 70 mg or about 140 mg in step ii), wherein said dose(s) is/are preferably given monthly.

### 2. Embodiments

2.1 A humanized monoclonal anti-calcitonin gene-related peptide (CGRP) antagonist antibody for use in a method of treating or preventing headache in a subject, wherein the method comprises:
i) selecting a subject who has been treated with a first dose of a humanized monoclonal anti-calcitonin gene-related peptide (CGRP) antagonist antibody, which subject has <50% reduction from baseline in Monthly Migraine Day (MMD) response (“MMD response”) after said first dose, and
ii) administering to the subject a second dose of a therapeutically effective amount of a humanized monoclonal anti-calcitonin gene-related peptide (CGRP) antagonist antibody,
   wherein said humanized monoclonal anti-calcitonin gene-related peptide (CGRP) antagonist antibody comprises a heavy chain variable region comprising the amino acid sequence set forth in SEQ ID NO: 6 and a light chain variable region comprising the amino acid sequence set forth in SEQ ID NO: 12.

2.2 The use according to Embodiment 2.1, wherein after said first dose of said CGRP antagonist antibody the subject has a less than 45%, less than 35%, less than 25%, less than 15%, less than 5% or not responding (≈0%) MMD response.

2.3 The humanized monoclonal anti-calcitonin gene-related peptide (CGRP) antagonist antibody for use in a method according to Embodiment 2.1 or 2.2, wherein the subject has migraine or cluster headache.

2.4 The humanized monoclonal anti-calcitonin gene-related peptide (CGRP) antagonist antibody for use in a method according to Embodiment 2.1, 2.2 or 2.3, wherein said migraine is episodic migraine, chronic migraine, episodic cluster headache or chronic cluster headache.

2.5 The humanized monoclonal anti-calcitonin gene-related peptide (CGRP) antagonist antibody for use in a method according to Embodiment 2.3 or 2.4, wherein the subject experiences migraines with aura.

2.6 The humanized monoclonal anti-calcitonin gene-related peptide (CGRP) antagonist antibody for use in a method according to Embodiment 2.3 or 2.4, wherein the subject experiences migraines without aura.

2.7 The humanized monoclonal anti-calcitonin gene-related peptide (CGRP) antagonist antibody for use in a method according to any one of the preceding Embodiments, wherein the subject in step i) of claim 1 has only received one dosage of said humanized monoclonal anti-calcitonin gene-related peptide (CGRP) antagonist antibody.

2.8 The humanized monoclonal anti-calcitonin gene-related peptide (CGRP) antagonist antibody for use in a method according to any one of the preceding Embodiments, wherein said subject received the same dosage in step i) and step ii) or a different dosage in step i) and step ii).

2.9 The humanized monoclonal anti-calcitonin gene-related peptide (CGRP) antagonist antibody for use in a method according to any one of the preceding Embodiments, wherein the monoclonal antibody is administered at a dose of about 100 mg to about 500 mg in step i) and at a dose of about 100 mg to about 500 mg in step ii).

2.10 The humanized monoclonal anti-calcitonin gene-related peptide (CGRP) antagonist antibody for use in a method according to any one of the preceding Embodiments, wherein the monoclonal antibody is administered at a dose of about 100 mg, about 300 mg or about 400 mg in step i) and at a dose of about 100 mg, about 300 mg or about 400 mg in step ii).

2.11 The humanized monoclonal anti-calcitonin gene-related peptide (CGRP) antagonist antibody for use in a method according to any one of the preceding Embodiments, wherein the monoclonal antibody is administered once per 3 months.

2.12 The humanized monoclonal anti-calcitonin gene-related peptide (CGRP) antagonist antibody for use in a method according to any one of the preceding Embodiments, wherein said selected subject experienced no MMD reduction following the first dose of said humanized monoclonal anti-calcitonin gene-related peptide (CGRP) antagonist antibody.

2.13 The humanized monoclonal anti-calcitonin gene-related peptide (CGRP) antagonist antibody for use in a method according to any one of the preceding Embodiments, wherein said selected subject did not experience a ≥50% MMD response to their first dose of said humanized monoclonal anti-calcitonin gene-related peptide (CGRP) antagonist antibody within 1 to 12 weeks.

2.14 The humanized monoclonal anti-calcitonin gene-related peptide (CGRP) antagonist antibody for use in a method according to any one of the preceding Embodiments, wherein said selected subject did not experience a ≥50% MMD response to their first dose of said humanized monoclonal anti-calcitonin gene-related peptide (CGRP) antagonist antibody within 1 to 4 weeks.

2.15 The humanized monoclonal anti-calcitonin gene-related peptide (CGRP) antagonist antibody for use in a method according to any one of the preceding Embodiments, wherein said selected subject did not experience a ≥50% MMD response to their first dose of said humanized monoclonal anti-calcitonin gene-related peptide (CGRP) antagonist antibody within 1 to 2 weeks.

### 3. Embodiments

3.1 Use of a humanized monoclonal anti-calcitonin gene-related peptide (CGRP) antagonist antibody for the manufacture of a medicament for use in a method of treating or preventing headache in a subject the method comprises:
i) selecting a subject who has been treated with a first dose of a humanized monoclonal anti-calcitonin gene-related peptide (CGRP) antagonist antibody, which subject has <50% reduction from baseline in Monthly Migraine Day (MMD) response (“MMD response”) after said first dose, and
ii) administering to the subject a second dose of a therapeutically effective amount of a humanized monoclonal anti-calcitonin gene-related peptide (CGRP) antagonist antibody,
   wherein said humanized monoclonal anti-calcitonin gene-related peptide (CGRP) antagonist antibody comprises a heavy chain variable region comprising the amino acid sequence set forth in SEQ ID NO: 6 and a light chain variable region comprising the amino acid sequence set forth in SEQ ID NO: 12.

3.2 The use according to Embodiment 3.1, wherein after said first dose of said CGRP antagonist antibody the subject has a less than 45%, less than 35%, less than 25%, less than 15%, less than 5% or not responding (≈0%) MMD response.

3.3 The use according to Embodiment 3.1 or 3.2, wherein the subject has migraine or cluster headache.

3.4 The use according to Embodiments 3.1, 3.2 or 3.3, wherein said migraine is episodic migraine, chronic migraine, episodic cluster headache or chronic cluster headache.

3.5 The use according to Embodiments 3.3 or 3.4, wherein the subject experiences migraines with aura.

3.6 The use according to Embodiment 3.3 or 3.4, wherein the subject experiences migraines without aura.

3.7 The use according to any one of the preceding Embodiments, wherein the subject in step i) of claim 1 has only received one dosage of said humanized monoclonal anti-calcitonin gene-related peptide (CGRP) antagonist antibody.

3.8 The use according to any one of the preceding Embodiments, wherein said subject received the same dosage in step i) and step ii) or a different dosage in step i) and step ii).

3.9 The use according to any one of the preceding Embodiments, wherein the monoclonal antibody is administered at a dose of about 100 mg to about 500 mg in step i) and a dose of about 100 mg to about 500 mg in step ii) of Embodiment 1.

3.10 The use according to any one of the preceding Embodiments, wherein the monoclonal antibody is administered at a dose of about 100 mg, about 300 mg or about 400 mg in step i) and about 100 mg, about 300 mg or about 400 mg in step ii) of Embodiment 1.

3.11 The use according to any one of the preceding Embodiments, wherein the monoclonal antibody is administered once per 3 months.

3.12 The use according to any one of the preceding Embodiments, wherein said selected subject experienced no MMD reduction following their first dose of said humanized monoclonal anti-calcitonin gene-related peptide (CGRP) antagonist antibody.

3.13 The use according to any one of the preceding Embodiments, wherein said selected subject did not experience a ≥50% MMD response to their first dose of said humanized monoclonal anti-calcitonin gene-related peptide (CGRP) antagonist antibody within 1 to 12 weeks of their first dose.

3.14 The use according to any one of the preceding Embodiments, wherein said selected subject did not experience a ≥50% MMD response to their first dose of said humanized monoclonal anti-calcitonin gene-related peptide (CGRP) antagonist antibody within 1 to 4 weeks of their first dose.

3.15 The use according to any one of the preceding Embodiments, wherein said selected subject did not experience a ≥50% MMD response to their first dose of said humanized monoclonal anti-calcitonin gene-related peptide (CGRP) antagonist antibody within 1 to 2 weeks of their first dose.

### EXPERIMENTAL SECTION

### Example 1

PROMISE-1 and PROMISE-2 were both phase 3, multicenter studies, and both had a double-blind, randomized, placebo-controlled, parallel-group design (Ashina et al. Cephalalgia 2020;40:241-54; Lipton et al. Neurology 2020;94:e1365-77). The PROMISE-1 study enrolled adults aged 18-75 years diagnosed with migraine at or before age 50 years and with a history of migraine for at least 1 year that included fewer than 15 headache days per month and at least four migraine days per month in the 3 months before screening. Following a 4-week screening period, patients were randomized in a 1:1:1:1 ratio to receive up to four treatments of eptinezumab 30 mg, 100 mg, or 300 mg, or placebo, administered intravenously on day 0, week 12, week 24, and week 36. The PROMISE-2 study enrolled adults aged 18-65 years diagnosed with migraine at ≤50 years of age who had a history of chronic migraine for ≥1 year before study screening. Following a 4-week screening period, patients were randomized in a 1:1:1 ratio to receive eptinezumab 100 mg, eptinezumab 300 mg, or placebo, administered intravenously on day 0 and at week 12. In both studies, change from baseline in MMDs over weeks 1 to 12 was the primary efficacy endpoint, and proportion of patients with ≥50% reduction in MMDs was a key secondary endpoint.

### Outcome Measures

Patients completed the headache electronic diary (eDiary) to document headache and migraine and completed several well-validated patient-reported outcome instruments. Two generic health-related quality of life instruments were completed in both PROMISE-1 and PROMISE-2: the 36-item Short-Form Health Survey (SF-36, v2.0) (Ware J, Kosinski M, Bjorner J, Turner-Bowker D, Gandek B, Maruish M. Development. User's Manual for the SF-36v2® Health Survey. Lincoln (RI): QualityMetric Incorporated; 2007) and the EuroQol 5-Dimensions 5-Levels visual analog scale (EQ-5D-5L VAS) (Herdman M, Gudex C, Lloyd A, et al. Development and preliminary testing of the new five-level version of EQ-5D (EQ-5D-5L). Qual Life Res. 2011;20(10):1727-1736). The Patient Global Impression of Change (PGIC)10 and patient-identified most bothersome symptom (PI-MBS) (Lipton et al., Headache. 2021 May;61(5):766-776; (Guy W (ed). ECDEU Assessment Manual for Psychopharmacology. Rockville, MD: US Department of Health, Education, and Welfare Public Health Service Alcohol, Drug Abuse, and Mental Health Administration, 1976) measures were completed in the PROMISE-2 study only. In addition, patients in PROMISE-2 completed the 6-item Headache Impact Test (HIT-6, v1.0) (Yang et al., Cephalalgia. 2011;31(3):357-367; Houts et al., Headache. 2020;60(9):2003-2013) which measures the impact of headaches on daily function and has been validated across episodic and chronic migraine populations (Kosinski et al. Qual Life Res 2003;12:963-974; Yang et al. Cephalalgia 2011;31:357-67). HIT-6 scores can range from 36 to 78, with an at-least 6-point decrease in score considered meaningful to patients with chronic migraine (Houts et al. Headache 2020;60:2003-13).

### Post Hoc Analysis

The current post hoc analysis was limited to patients who were suboptimal responders over weeks 1-12 and who had patient-reported outcome data available at weeks 12 and 24. Suboptimal responders were defined as patients with a <50% reduction from baseline in MMDs; responders were defined as patients with a ≥50% reduction from baseline in MMDs. Data from the two studies were analyzed separately. For each study, data from the eptinezumab 100 mg and 300 mg dose arms were pooled. The eptinezumab 100 mg and 300 mg doses have generally similar efficacy and are expected to behave similarly, and pooling provided a larger sample size for increased precision of estimation. The following clinically helpful or common practice endpoints, measured over weeks 1 to 12, were assessed as potential predictors of response status over weeks 13 to 24, using logistic regression analysis: percent change in MMDs, change in SF-36 scale scores, change in EQ-5D-5L VAS, and change in percent of severe pain headache. In addition, the following were evaluated for PROMISE-2 only: change in HIT-6 total score, PGIC, and change in severity of PI-MBS. The full model included all potential predictor variables at week 12/weeks 1-12. A stepwise procedure was used to identify parameters. The stepwise selection procedure used 5% entry and 10% retention criteria.

### RESULTS

### Second Dose Responders

Overall, 416 patients (46.8% of treated participants) in the PROMISE-1 study and 479 patients (44.7% of treated participants) in the PROMISE-2 study had a <50% MMD response across weeks 1-12 and had patient-reported outcome data available at weeks 12 and 24. In PROMISE-1, the proportion of suboptimal responders to the first eptinezumab infusion across weeks 1-12 who were responders to the second eptinezumab infusion across weeks 13-24 was 38.0% (38/100), 37.6% (38/101), and 36.3% (33/91) with eptinezumab 30 mg, 100 mg, and 300 mg, respectively, and was 37.0% (71/192) for the pooled eptinezumab 100 mg and 300 mg doses. The corresponding rate with placebo was 33.9% (42/124). In PROMISE-2, the proportion of suboptimal responders to the first eptinezumab infusion across weeks 1-12 who were responders to the second eptinezumab infusion across weeks 13-24 was 28.7% (41/143), and 29.0% (38/131) with eptinezumab 100 mg and 300 mg, respectively, and was 28.8% (79/274) for the pooled eptinezumab dose levels. The corresponding rate with placebo was 18.5% (38/205). The HIT-6 responder rate (≥6-point score decrease) at week 12 in PROMISE-2 overall was 47.2% (168/356) and 56.0% (196/350) with eptinezumab 100 mg and 300 mg, respectively, and was 36.3% (133/366) with placebo. In first-dose suboptimal responders, the HIT-6 responder rate at week 12 was 22.5% (34/151) and 29.6% (40/135) with eptinezumab 100 mg and 300 mg, respectively, and was 23.4% (52/222) with placebo.

### First Dose Predictors of Second Dose Response

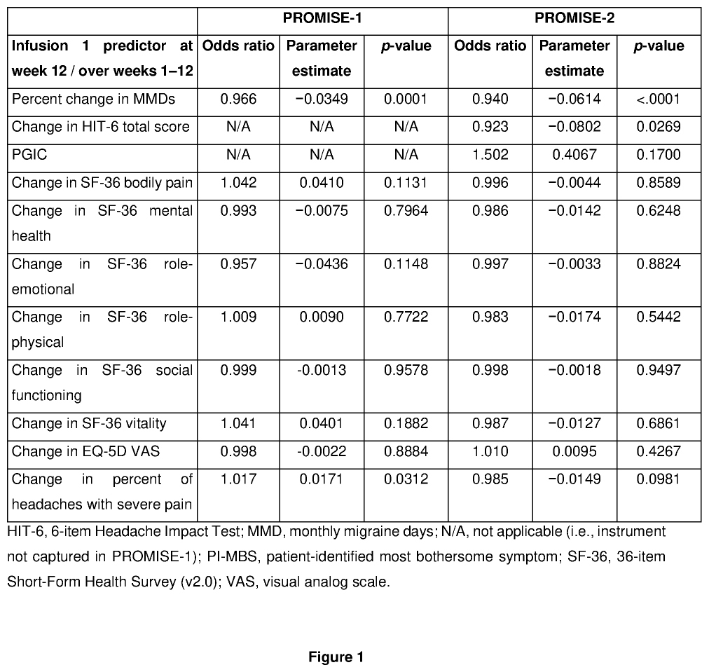

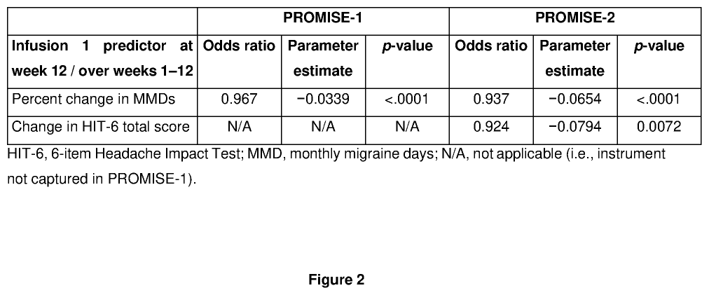

The full logistic regression analysis (model including all possible predictors) of pooled data from the eptinezumab 100 mg and 300 mg treatment groups demonstrated that percent change in MMDs across weeks 1-12 was a significant first-infusion predictor of second-infusion ≥50% MMD response in the PROMISE-1 and PROMISE-2 studies; change in HIT-6 total score, which was assessed only in the PROMISE-2 study, was an additional significant first dose predictor of second-infusion response (FIG. 1). Thus, the final stepwise logistic regression analysis included one predictor for PROMISE 1 (percent change in MMDs) and two predictors for PROMISE-2 (percent change in MMDs and change in HIT-6 total score) (FIG. 2).

### Probability of Second Dose Response Based on First Dose Predictors

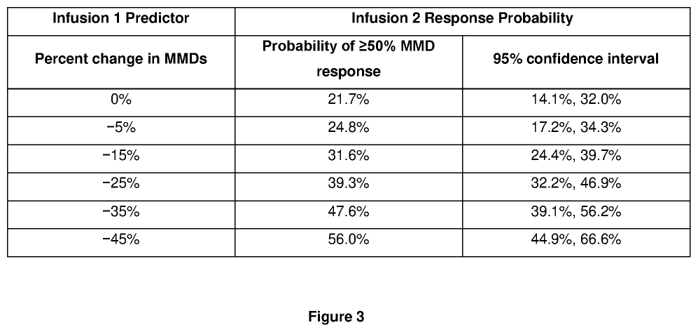

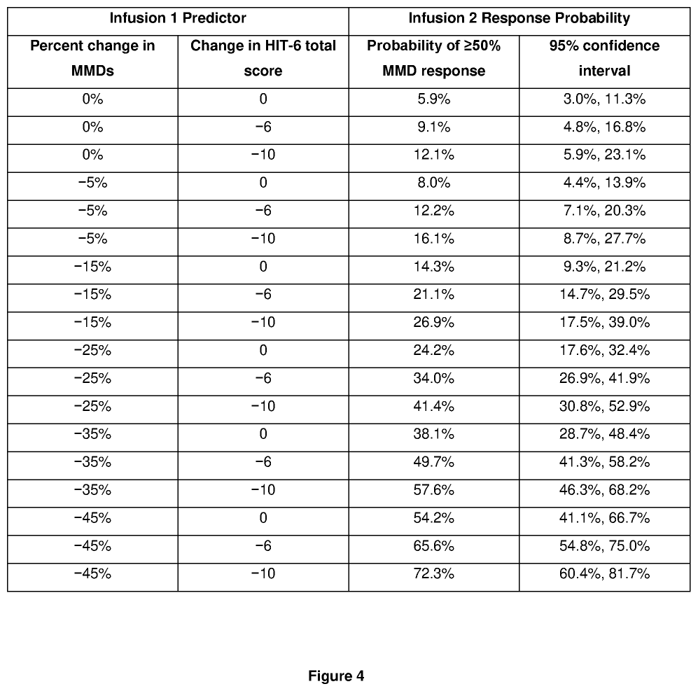

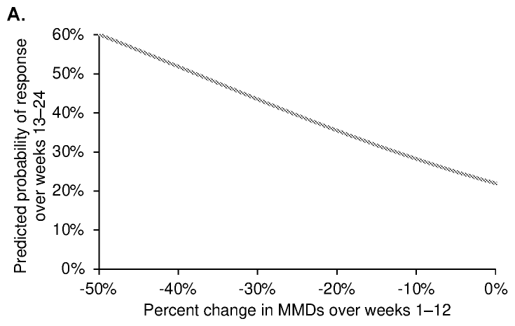

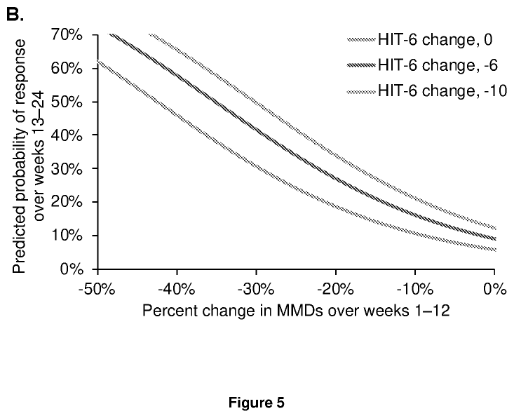

Stepwise modelling results of probability of second dose response based on first dose predictors in the PROMISE-1 and PROMISE-2 studies are shown in FIGS. 3 and 4, respectively, and are presented graphically in FIG. 5. The equation for PROMISE-1 is

$${P}_{50 \%} = \frac{1}{1 + {e}^{- ( - 1.2803 - 0.0339 * {Δ}_{1} )}} ,$$

where P_{50%} is the probability of being a responder in infusion 2 and Δ₁ is the percent change in MMDs over infusion 1 (e.g., a 30% reduction in MMDs over infusion 1 results in Δ₁=−30).

The equation for PROMISE-2 is

$${P}_{50 \%} = \frac{1}{1 + {e}^{- ( - 2.7747 - 0.0654 * {Δ}_{1} - 0.0794 * {H}_{1} )}} ,$$

where P_{50%} is the probability of being a responder in infusion 2 and Δ₁ is the percent change in MMDs over infusion 1 and H₁ is the change in HIT-6 score from baseline to the end of infusion 1 (e.g., a 6-point reduction in HIT-6 total score over infusion 1 results in H₁=−6).

1. A method of treating or preventing headache in a subject, wherein the method comprises:
i) selecting a subject who has been treated with a first dose of a humanized monoclonal anti-calcitonin gene-related peptide (CGRP) antagonist antibody, which subject has a <50% reduction from baseline in Monthly Migraine Days (“MMD response”) after said first dose, and
ii) administering to the subject a second dose, said second dose comprising a therapeutically effective amount of said humanized monoclonal anti-calcitonin gene-related peptide (CGRP) antagonist antibody,
wherein said humanized monoclonal anti-calcitonin gene-related peptide (CGRP) antagonist antibody in (i) and (ii) comprises a heavy chain variable region comprising the amino acid sequence set forth in SEQ ID NO: 6 and a light chain variable region comprising the amino acid sequence set forth in SEQ ID NO: 12.

2. A method of treating or preventing headache in a subject, which method comprises:
i) determining whether a subject who has been treated with a first dose of a humanized monoclonal anti-calcitonin gene-related peptide (CGRP) antagonist antibody has a <50% reduction from baseline in Monthly Migraine Days (“MMD response”) after said first dose, and
ii) administering to the subject a second dose of said humanized monoclonal anti-calcitonin gene-related peptide (CGRP) antagonist antibody, said second dose comprising a therapeutically effective amount of said humanized monoclonal anti-calcitonin gene-related peptide (CGRP) antagonist antibody, if the subject has been determined to have <50% reduction from baseline in MMD response after said first dose,
wherein said humanized monoclonal anti-calcitonin gene-related peptide (CGRP) antagonist antibody comprises a heavy chain variable region comprising the amino acid sequence set forth in SEQ ID NO: 6 and a light chain variable region comprising the amino acid sequence set forth in SEQ ID NO: 12.

3. The method according to claim 1 or 2, wherein after the first dose of said CGRP antagonist antibody the subject has a less than 45%, less than 35%, less than 25%, less than 15%, less than 5% or not responding (≈0%) MMD response.

4. The method according to claim 1, 2 or 3, wherein the subject has migraine or cluster headache.

5. The according to claim 1, 2, 3 or 4, wherein said headache is episodic migraine, chronic migraine, episodic cluster headache or chronic cluster headache.

6. The method according to claim 4 or 5, wherein the subject experiences migraines with aura.

7. The method according to claim 4 or 5, wherein the subject experiences migraines without aura.

8. The method according to any one of the preceding claims, wherein the subject in step i) of claim 1 or 2 has only received one dose of said humanized monoclonal anti-calcitonin gene-related peptide (CGRP) antagonist antibody.

9. The method according to any one of the preceding claims, wherein said subject received the same dose in step i) and step ii) or a different dose in step i) and step ii).

10. The method according to any one of the preceding claims, wherein the monoclonal antibody is administered at a dose of about 100 mg to about 500 mg in step i) and a dose of about 100 mg to about 500 mg is administered in step ii) of claim 1 or 2.

11. The method according to any one of the preceding claims, wherein the monoclonal antibody is administered at a dose of about 100 mg, about 300 mg or about 400 mg in step i) and about 100 mg, about 300 mg or about 400 mg is administered in step ii) of claim 1 or 2.

12. The method according to any one of the preceding claims, wherein the monoclonal antibody is administered about once every 3 months.

13. The method according to any one of the preceding claims, wherein said selected subject did not experience any MMD reduction after their first dose of said humanized monoclonal anti-calcitonin gene-related peptide (CGRP) antagonist antibody.

14. The method according to any one of the preceding claims, wherein said selected subject did not experience a ≥50% MMD response to their first dose of said humanized monoclonal anti-calcitonin gene-related peptide (CGRP) antagonist antibody within 1 to 12 weeks after the first dose.

15. The method according to any one of the preceding claims, wherein said selected subject did not experience a ≥50% MMD response to their first dose of said humanized monoclonal anti-calcitonin gene-related peptide (CGRP) antagonist antibody within 1 to 4 weeks after the first dose.

16. The method according to any one of the preceding claims, wherein said selected subject did not experience a ≥50% MMD response to their first dose of said humanized monoclonal anti-calcitonin gene-related peptide (CGRP) antagonist antibody within 1 to 2 weeks after the first dose.

17. The method according to any one of the preceding claims, wherein if the subject has a ≥50% MMD response after administration of the second dose, the subject is administered a third dose of said humanized monoclonal anti-CGRP antagonist antibody, wherein the third dose of said humanized monoclonal anti-CGRP antagonist antibody optionally is the same or greater than the first or second antibody dose amounts.

18. The method according to any one of the preceding claims, wherein if the subject does not have a ≥50% MMD response after administration of the second dose, the subject is administered a third dose of said humanized monoclonal anti-CGRP antagonist antibody, wherein the dose amount of said third dose of humanized monoclonal anti-CGRP antagonist antibody optionally is greater than the first or second antibody dose amounts.

19. The method according to any one of the preceding claims, wherein if the subject does not have a ≥50% MMD response after administration of the second dose, then the subject is not administered a third dose of said humanized monoclonal anti-calcitonin gene-related peptide (CGRP) antagonist antibody.
